(19) United States
(12) Patent Application Publication
(10) Pub. No.: US 2010/0212335 A1
(43) Pub. Date: Aug. 26, 2010

(54) Energy Saving and Environmentally Friendly Mobile Atmospheric Dehumidifier For Water Generator and Drinking Purposes

(75) Inventor: Arda Rahardja Lukitobudi, Cimahi (ID)

Correspondence Address: SWANSON & BRATSCHUN, L.L.C., 8210 SOUTHPARK TERRACE, LITTLETON, CO, 80120, US

(73) Assignee: ETERNAIR WATER PTE. LTD., Singapore (SG)

(21) Appl. No.: 12/682,399

(22) PCT Filed: Oct. 10, 2007
(86) PCT No.: PCT/SG2007/000342

§ 371 Date: Apr. 9, 2010

(51) Int. Cl. F25D 17/06; F25D 27/00; F25D 21/14; F25B 41/00

(52) U.S. Cl. 62/93; 62/264; 62/291; 62/513

(57) ABSTRACT

An atmospheric dehumidifier air handling unit producing condensation water from moisture in the atmospheric suitable for water generator and drinking purposes. A method and apparatus to produce pure condensation water from moisture in the atmosphere using energy saving and environmentally friendly atmospheric dehumidifier of a condensing unit. In warm climates, filtered and sterilized fresh atmospheric air is passed through several evaporator cooling coils to condense the moisture in the atmospheric air. The condensed water is then collected on a drip pan and into a discharge line cooler. In cool climates, filtered and sterilized cold fresh atmospheric air is passed to a condenser of the condensing unit of the air handling unit. The fresh heated air is then passed to several evaporator cooling coils to condense the moisture in the atmospheric air. The condensed water is then collected on a drip pan.

### FIELD OF THE INVENTION

The present invention relates to water purification units.

### BACKGROUND TO THE INVENTION

Worldwide water shortage is a crisis. Many countries of the world already have an inadequate water supply. In the past, people searched for locations to settle wherever freshwater sources were conveniently accessible, nearby rivers or lakes, and around locations of abundant groundwater or within regions of sufficient rainfall. Whenever water resources became scarce, they moved on to develop new settlements where they had easy access of water supply. As the earth became heavily populated, fertile and water-rich land became overcrowded and eventually communities had to settle in regions of limited water resources. Usable water supplies have been reduced by pollution and sewage waste. Furthermore, change in weather patterns has reduced rainfall in some areas and deserts started to creep in as water resources were depleted, while the inhabitants had no recourse but to stay. Water treatment such as with chemicals such as chlorine or other halogens, however, the by-products of such treatment may be toxic and the result in further contamination. Treated municipal water supplies may be contaminated with may lead to health problems in drinking such water.

These days many commercial potable water sources are produced from the ground water. The ground water might be contaminated by pollutants such as pesticides or chemical wastes. Taking the ground water in a large scale may lead to severe problems such as decreasing the ground level and shortage of needed water supplies or other environmental problems. Various attempts have been made to come up with a system for dehumidifying moisture of air and purifying the water produced in such systems for drinking purposes. Producing potable water by dehumidifying moisture from the air, is an attractive alternative.

In one prior art, the unit also must be primed initially with approximately 10 litres of start-up water which can be a source of initial contaminants, such as volatile organic compounds (VOC), which are neither removed nor broken down by either UV radiation or granular carbon charcoal. The compressor operates to maintain a cold set-point temperature within the water reservoir, i.e., the compressor operates to cool the fluid remaining in the reservoir even when the device is not actively producing water condensate.

This may also be achieved through the use of a refrigeration system including a compressor, evaporator, fan condenser, and a reservoir system provided as an indoor unit, window or wall mounted unit, or a portable or mobile indoor/outdoor unit, and may be integrated with an existing air conditioning system and/or a conventional refrigerator, or mounted in a vehicle. The apparatus may also function as a dehumidifier, an air purifier, or a heat pump for cooling or heating air. In some embodiments the compressor, condenser, and water dispenser are remote from the housing. Collected water may be cooled by a secondary evaporator and heated by a secondary condenser or strip heater. The secondary condenser and secondary evaporator may be connected with a secondary compressor to isolate the system for cooling water from that of condensing the air. Such units are often befouled by bacteria or other contaminants sometimes caused from insects or rodents entering the units.

Similar prior art disclose systems and methods for extraction of freshwater from ambient air in regions of extremely hot and humid climates, for supply of drinking water and freshwater for small to large groups of people in areas that freshwater resources are not conveniently accessible. Compact mobile units are disclosed to provide freshwater and drinking water for resort areas, to passengers on land and sea vehicles, in situations of emergency, and to areas of water shortage. The art of adaptation of commercial dehumidification units is taught in design and construction of apparatus for production of freshwater and drinking water. Preparation of drinking water included ultraviolet disinfection, ozone treatment, and/or chlorine addition; activated carbon and ion exchange filters; and adding of fluorine, air/oxygen to refresh the water storage units, and minerals for taste and health provisions. For energy economy and use of independent electric power supply, the produced water may flow under gravitational forces entirely or with the assistance of small boasting pumps. Lukewarm, hot, cold, and/or cold carbonated drinking water are provided as well as freshwater for other usage.

Many of the systems available present maintenance problems relating to the positioning of drains.

The prior art commonly use a typical refrigerant deicer system to keep their evaporators from freezing under low condensate flow rates, which can occur with cool ambient air. The units usually use large capacity refrigerant gas dehumidifiers. The refrigerant gas from the compressor cools an evaporator coil and, when ambient air is passed by the coil, moisture condenses out and drips to a collector below. When operated over extended periods or in cooler temperatures, the evaporator tends to freeze over due to low flow rate of condensate. Some have overcome this problem by switching over to hot-gas bypass mode. A thermostat and/or humidistat control assists in determining when the compressor switches over. When the temperature of the incoming air is too low, this on/off cycle during cooler temperatures drastically reduces production of water until the compressor eventually stops.

There is thus a need for a unit that can operate in a wide range of conditions including hot weather or cold weather. Such a device should be movable and the water extracted from the humidity taken out of the ambient air should be potable.

It is an object of this invention to provide a dehumidifier for dehumidifying moisture of the air while purifying the water extracted from the humidity making it potable for drinking purposes.

### SUMMARY OF THE INVENTION

Throughout this document, unless otherwise indicated to the contrary, the frame “comprising”, “consisting of”, and the like, are to be construed as inclusive and not exhaustive.

In accordance with a first aspect of the invention there is a method for dehumidifying an atmospheric air or the like comprising the steps of:
(a) drawing the atmospheric air across a cooling means thereby cooling the atmospheric air; and
(b) at the same time rejecting the heat of the atmospheric air to remove moisture therefrom;
(c) transferring the moisture removed from the atmospheric air or the like to a filtering system thereby
(d) filtering and purifying the removed moisture wherein the purified water is suitable for a water generator or drinking purposes.

The step of drawing the atmospheric air comprises passing the atmospheric air through a duct of an Air Handling Unit.

The step of passing the atmospheric air through a series of an air pre filter, a medium filter and a set of UV lights.

The cooling means is an evaporator coil.

The direction of drawing the atmospheric air can be switched in a reverse direction using a blower such that in a warm climate the atmospheric air is passed through the cooling means then heated while in a cool climate, the atmospheric air is heated then passed through the cooling means.

The step of heating the atmospheric air is conducted by a condenser.

The step of cooling the atmospheric air produces condensation formed from the moisture in the atmospheric air, the condensation is thereby collected on a drip pan and transferred to a discharge line cooler wherein the condensation will pass over a refrigerant discharge line of a condensing unit.

The condensation is pumped to a storage tank further comprising the step of monitoring the level of water in the storage tank and drawing the atmospheric air across the cooling means when the level reaches a predetermined lower level in the storage tank.

The cooling of the atmospheric air is achieved by connecting the evaporator coil with an expansion valve and the condensing unit.

The condensing unit consist of compressors, condenser, liquid receiver, liquid line sight glass and liquid line filter dryer.

An environmentally friendly refrigerant is used in the refrigerant line of the condensing unit, evaporator coils and expansion valve.

The cooling means and the condenser are in close proximity thereby reducing energy needed for the method.

The refrigerant discharge line is dipped into the discharge line cooler.

The atmospheric air is then being heated by the condenser or in other words, the condenser is then cooled by the cold air coming from the cooling means of the Air Handling Unit.

An apparatus for dehumidifying atmospheric air or the like comprising:
a housing;
an atmospheric air intake means mounted in the housing;
a cooling means mounted in the housing;
an air pre filter, a medium filter and a set of UV lights;
a heat-exchanging mean mounted in the housing;
a chamber associated with the housing;

The apparatus includes a panel mounted on the housing for operating the apparatus, including an inverter adapter mounted on the housing.

A dehumidifier and water condensation system for dehumidifying moisture or the like comprising:
a) a dehumidifier for holding moisture or the like;
b) a humidity removal means for drawing humid ambient air from the atmosphere for removing humidity from the ambient air thereby forming dry heated air; and
c) a dispensing means for dispensing said dry heated air to the dehumidifier to thereby dehumidify the moisture or the like and remove moisture therefrom;
   including the step of recovering moisture from the ambient air after dehumidifying the same and purifying the moisture to form a condensation water.

### BRIEF DESCRIPTION OF THE DRAWINGS

FIG. 1 is a diagrammatic view illustrating the flow process of the system.

### DETAILED DESCRIPTION OF THE PREFERRED EMBODIMENTS

Described is an atmospheric dehumidifier air handling unit producing condensation water from moisture in the atmosphere suitable for a water generator and drinking purposes.

A Method and apparatus to produce pure condensation water from moisture in the atmosphere using energy saving and environmentally friendly atmospheric dehumidifier of a condensing unit comprising:

In warm climates, filtered and sterilized fresh atmospheric air is passed through several evaporator cooling coils to condense the moisture in the atmospheric air. The condensed water is then collected on a drip pan and into a discharge line cooler. To reduce the power consumption, the cold air after passing the evaporator cooling coils is then passed to the condenser of the condensing unit of the air handling unit. A refrigerant discharge line coming out from the compressor is dipped through the discharge line cooler of cold condensation drip of the water to gain further energy saving.

In cool climates, filtered and sterilized cold fresh atmospheric air is passed to a condenser of the condensing unit of the air handling unit. The fresh heated air is then passed to several evaporator cooling coils to condense the moisture in the atmospheric air. The condensed water is then collected on a drip pan. The refrigerant discharge line coming out from the compressor is also dipped through the discharge line cooler of cold condensation drip of the water.

The system is additionally fitted with an alternative environmentally friendly refrigerant to further reduce power consumption.

In accordance with a first aspect of the invention there is a method for dehumidifying an atmospheric air or the like comprising the steps of
(a) drawing the atmospheric air across a cooling means thereby cooling the atmospheric air;
(b) removing moisture from the atmospheric air;
(c) transferring the moisture removed from the atmospheric air or the like to a filtering system
(d) filtering and purifying the removed moisture wherein the purified water is suitable for a water generator or drinking purposes.

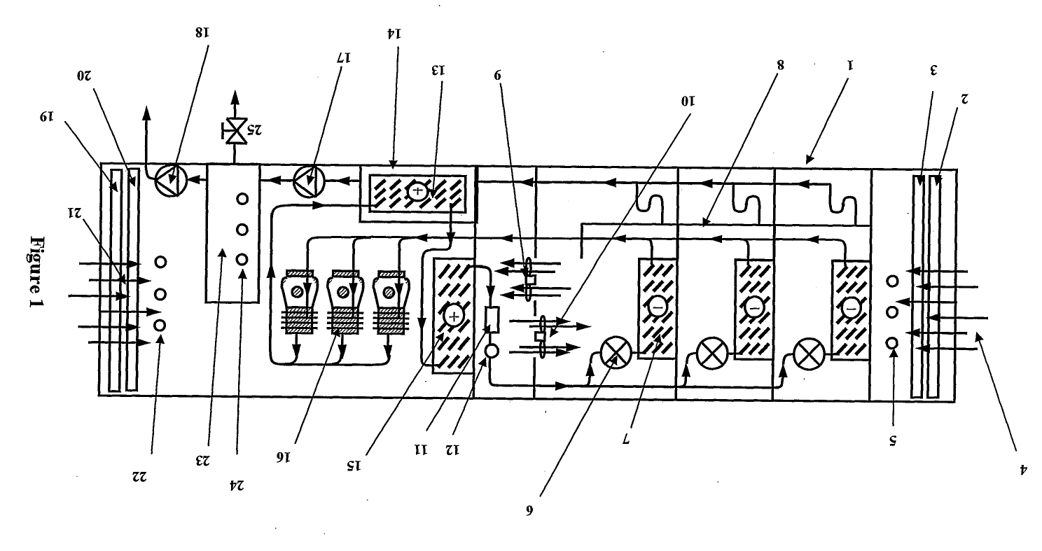

Referring now to FIG. 1 of the drawings, the following description will describe in detail an energy saving and environmentally friendly potable water production dehumidifier. There are two schemes of operation, which the unit can be operated either in summer and autumn in subtropical or tropical countries where the direction of the drawn air is passed through the evaporator coils then to the condenser; while in winter and spring, the direction of the drawn air is passed through the condenser then to the evaporator coils.

In the first scheme of operation, the compressor (16) will compress the refrigerant vapour to the condenser (15) through discharge line (13) in high pressure and temperature. The discharge line (13) is cooled by dipping it into the cold condensation water drop from the evaporator coils (7) in the discharge line cooler (14), which is collected from the drip pan (8), so it will save the energy. In the condenser (15), the refrigerant vapour will reject heat causing the refrigerant being condensed. After passing the condenser (15), then the liquid refrigerant will pass to the expansion valves (6) through the liquid line filter dryer (11) and liquid line sight glass (12). After the refrigerant being expanded in the expansion valves (6) then the refrigerant pressure and temperature will drop significantly and enter to the evaporator coils (7).

In the dehumidifier evaporator coils (7), the liquid refrigerant will absorb heat of the fresh air (4) and it will evaporate, so it will cool the fresh air (4) that contains moisture, so that the moisture of the fresh air will condense. After passing the evaporator coils (7), the refrigerant vapour will return to the compressor (16). While the atmospheric outdoor fresh air (4) is withdrawn into the air handling unit duct (1) by the blower (9), the outdoor warm fresh air (4) will enter the air pre filter (2) and medium filter (3) for dust, dirt, and other pollutants to be eliminated. The fresh air also passes through the UV lights (5) to sterilize the air. Then the fresh air (4) is passed to the dehumidifier evaporator coils (7). In the dehumidifier evaporator coils (7), the moisture of the fresh air (4) will condense. The drip of the condensation will be collected to the drip pan (8) and then will be passed to the discharge line cooler (14) where it will cool the discharge line (13). The condensed water in the discharge line cooler will be pumped by condensed water pump (17) to the holding tank (23). The water in the holding tank can be pumped by holding tank pump (18) to be used to any application or just by gravity by opening the valve (25). The cold fresh air after passing the evaporator coils (7) will flow to the condenser (15) and cool it so as to save energy. After passing the condenser (15), the exhaust air will flow to the outdoor (21) through the UV lights (24), air medium filter (20) and pre filter (19).

It is important to control the temperature so that the fresh air is cooled at the dew point and the moisture will condense at this point. It is also necessary to control the refrigerant pressure to a minimum and maximum allowable pressure to maintain the compressor (16) in a safe condition. Oil pressure control is also necessary to make sure that the oil is always present in the compressor (16). If the air pre and medium filters are dirty and need to be changed, it is necessary to have a control to make a sign that those filters should have to be changed.

In the scheme of winter operation, the compressor (16) will compress refrigerant vapour to the condenser (15) through discharge line (13) in high pressure and temperature. The discharge line (13) is cooled by dipping it into the cold condensation water drop from the evaporator coils (7) in the discharge line cooler (14), which is collected from the drip pan (8), so it will save energy. In the condenser (15), the refrigerant vapour will reject heat causing the refrigerant being condensed. After passing the condenser (15), then the liquid refrigerant will pass to the expansion valves (6) through the liquid line filter dryer (11) and liquid line sight glass (12). After the refrigerant being expanded in the expansion valves (6) then the refrigerant pressure and temperature will drop significantly and enter to the evaporator coils (7). In the dehumidifier evaporator coils (7), the liquid refrigerant will absorb heat of the heated fresh air and it will evaporate, so it will cool the heated fresh air (21) that contains moisture, so that the moisture of the fresh air will condense. After passing the evaporator coils (7), the refrigerant vapour will return to the compressor (16). While the atmospheric outdoor cold fresh air (21) is withdrawn into the Air Handling Unit duct (1) by the blower (10), the outdoor fresh air (21) will enter the air pre filter (19) and medium filter (20) for dust, dirt, and other pollutants to be eliminated. The fresh air also passes through the UV lights (22) to sterilize the air. Then the cold fresh air (21) is passed to the condenser (15) to be heated so that the humidity will be increased. After passing the condenser (15), the heated fresh air will flow to the dehumidifier evaporator coils (7). In the dehumidifier evaporator coils (7), the moisture of the heated fresh air will condense. The drip of the condensation will be collected to the drip pan (8) and then will be passed to the discharge line cooler (14) where it will cool the discharge line (13). The condensed water in the discharge line cooler will be pumped by cooler pump (17) to the holding tank (23). The water in the holding tank can be pump by tank pump (18) to be used to any application or just by gravity by opening the valve (25). After passing the evaporator coils (7), the cold fresh air will flow to the outdoor (4) through the UV lights (5), air medium filter (3) and pre filter (2).

The water production machine of this invention consist of:

Air handling unit (1); several evaporator coils (7) to condense the moisture; drip pan (8) to each evaporator coil (7) to collect the water condensation; Where the air handling unit (1) is connected to fresh air, in the intake (4) of the air handling unit is installed the air pre filter (2), medium filter (3) and UV lights (5).

In the air handling unit (1) is installed several evaporator coils (7) to condensate the moisture, and at the bottom of the air handling unit below each evaporator coil is installed drip pan (8) of the condensate which is generate from each coil, and a water pipe to collect the water which is connected to a discharge line cooler (14) and then supplied to a water holding tank/storage tank (23) which has UV lights (24). The air then flows to the outdoor (21) through the condenser (15), UV lights (22), air pre filter (20) and medium filter (19).

The water production machine of this invention using brine as a cooling medium and an environmentally friendly refrigerant is to gain environmentally friendly substance application. All parts, including pipes and tanks which the potable water flows in, is made of food grade material, preferably stainless steel.

An energy saving dehumidifier consists of : condenser (15) which is cooled by the cold air after passing the evaporator coils (7); the discharge line (13) which is dipped to a cold condensate water which is produced by the evaporator coils; the refrigerant will be subcooled, then flows through the liquid receiver, liquid line filter dryer, liquid line sight glass, and to the expansion valves (6), and after expanded by the expansion valves then flows to the evaporator coils (7), and return to the compressor (16), then after compressed by the compressor, the refrigerant flows to the condenser. The work of the compressor is decreased due to the handling of the cooling medium of the condenser which will save energy.

A combination of an energy saving and environmentally friendly dehumidifier and a water generator which consists of: a water generator machine using evaporator coils of a dehumidifier; and condensate water of evaporator coils (7) to cool the discharge line; where the refrigerant of the cooled condenser (15) are passed to the dehumidifier evaporator coils (7) through the expansion valves, which will cool the air and condensate the moisture of the air. The condensate will drip and collected to a pan (8) and circulate to a holding tank/storage tank (23) after passing the discharge line cooler (14).

A method to produce condensate water from fresh air using energy saving and environmentally friendly dehumidifier, comprising of the following steps: For summer and autumn operation, the fresh air (4) flows through air pre filter (2), medium filter (3) and UV lights (5), then passed to several evaporator coils (7) to condensate the moisture and the condensate collected to a pan (8). Passing the discharge line (13) by dipping it to a discharge line cooler (14) by cold condensate water, so the refrigerant after the condenser will be subcooled and after passing through the expansion valve, will cool the air in the evaporator coils (7) to condensate the moisture passing through the evaporator coils (7). The cold air flows to the condenser (15) and then to the outdoor through the UV lights (22), air medium filter (20) and pre filter (19).

While for winter and spring operation, the fresh air (21) flows through air pre filter (19), medium filter (20) and UV lights (22), then passed to the condenser (15) to heat the air, increasing the humidity of the air. Then the air is passed to several evaporator coils (7) to condensate the moisture and the condensate collected to a pan (8). Passing the discharge line (13) by dipping it to a discharge line cooler (14) by cold condensate water, so the refrigerant after the condenser will be subcooled and after passing through the expansion valve, will cool the air in the evaporator coils (7) to condensate the moisture passing through the evaporator coils (7). The cold air flows to the outdoor through the UV lights (5), air medium filter (3) and pre filter (2).

While the necessary sensors are sensing the parameter of as follows : discharge pressure, suction pressure, discharge temperature, suction temperature, outdoor air temperature and relative humidity, before evaporator coil air temperature and relative humidity, after evaporator coil air temperature and relative humidity, room air temperature and relative humidity, etc. The electrical sensors are as follows : electrical voltage, electrical frequency, electrical motor cos phi, electrical motor rpm, compressor electrical current, condenser fan electrical current, air handling unit blower electrical current, total electrical current, ultraviolet lamp indicator, compressor lamp indicator, condenser fan lamp indicator, air handling unit blower lamp indicator, system failure lamp indicator , etc.

Although a particular embodiment of the invention has been disclosed, modifications and variations as would be apparent to a skilled addressee are deemed to be within the scope of the present invention.

1. A method for dehumidifying an atmospheric air or the like, the method comprising:
passing the atmospheric air through a series of an air pre filter, a medium filter and a set of UV lights;
drawing the atmospheric air across a cooling means thereby cooling the atmospheric air;
at the same time rejecting the heat of the atmospheric air to remove moisture therefrom;
transferring the moisture removed from the atmospheric air or the like to a filtering system thereby; and
filtering and purifying the removed moisture wherein the purified moisture is suitable for a water generator or drinking purposes.

2. The method according to claim 1 wherein the step of drawing the atmospheric air comprises passing the atmospheric air through a duct of an Air Handling Unit.

3. The method according to claim 1, the cooling means is an evaporator coil.

4. The method according to claim 1, wherein the direction of drawing the atmospheric air can be switched in a reverse direction using a blower such that in a warm climate the atmospheric air is passed through the cooling means then heated while in a cool climate, the atmospheric air is heated then passed through the cooling means.

5. The method of claim 1, wherein the step of heating the atmospheric air is conducted by a condenser.

6. The method according to claim 1, wherein the step of cooling the atmospheric air produces condensation formed from the moisture in the atmospheric air, the condensation is thereby collected on a drip pan and transferred to a discharge line cooler wherein the condensation will pass over a refrigerant discharge line of a condensing unit.

7. The method according to claim 6 wherein the condensation is pumped to a storage tank further comprising the step of monitoring the level of water in the storage tank and drawing the atmospheric air across the cooling means when the level reaches a predetermined lower level in the storage tank.

8. The method according to claim 6. wherein the cooling of the atmospheric air is achieved by connecting the evaporator coil with an expansion valve and the condensing unit.

9. The method of claim 6, wherein the condensing unit comprises one or more compressors, a condenser, a liquid receiver, a liquid line sight glass, and a liquid line filter dryer.

10. The method according to claim 8, wherein an environmentally friendly refrigerant is used in the refrigerant line of the condensing unit, evaporator coils and the expansion valve.

11. The method according to claim 5, wherein the cooling means and the condenser are in close proximity thereby reducing energy needed for the method.

12. The method according to claim 6, wherein the refrigerant discharge line is dipped into the discharge line cooler.

13. The method according to claim 2, wherein the atmospheric air is heated by the condenser or in other words, the condenser is cooled by the cold air coming from the cooling means of the Air Handling Unit.

14. An apparatus for dehumidifying atmospheric air or the like_{:}
the system comprising:
a housing;
an atmospheric air intake means mounted in the housing;
a cooling means mounted in the housing;
a series of an air pre filter, a medium filter and a set of UV lights positioned upstream to the atmospheric air intake means;
a heat-exchanging mean mounted in the housing; and (f) a chamber associated with the housing;

15. The apparatus according to claim 14 including a panel mounted on the housing for operating the apparatus, including an inverter adapter mounted on the housing.

16. A dehumidifier and water condensation system for dehumidifying moisture or the like. the system comprising:
an air filtering means comprising a series of air pre filter, medium filter and a set of UV lights for filtering and sterilizing inlet humid ambient air;
a drawing means for drawing humid ambient air from the atmosphere and the air filtering means is positioned upstream to the drawing means;
a humidity removal means for removing humidity from the humid ambient air thereby forming dry heated air;
a condensation means for condensing the humidity removed from the humidity removal means thereby forming condensed water; and
a discharging means for discharging the condensed water from the condensation means to a water collection means.

17. The method of claim 1, wherein filtering and purifying the removed moisture comprises filtering and purifying the removed moisture with one or more water filters and one or more ultraviolet light tubes.

18. The system of claim 16, wherein the discharging means discharges the condensed water to a water collection means via one or more water filters and one or more ultraviolet light tubes.

19. The method of claim 6, further comprising, after passing the discharge line cooler, passing the condensation through one or more water filters and one or more UV light tubes.

20. The method of claim 6, wherein the drip pan, the discharge line cooler, and the storage tank are made from food grade stainless steel.
